(19) United States
(12) Patent Application Publication
(10) Pub. No.: US 2010/0077538 A1
(43) Pub. Date: Apr. 1, 2010

(54) Swimming goggles

(76) Inventor: Herman Chiang, Chung-Ho City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 12/285,970

(22) Filed: Oct. 17, 2008

(30) Foreign Application Priority Data
Sep. 30, 2008 (TW) 97217568

(51) Int. Cl. A61F 9/02

(52) U.S. Cl. 2/428; 2/445; 2/452

(57) ABSTRACT

Swimming goggles include a left frame, a right frame, hard enveloping frames, soft pads and a head strap. Each of the left frame and the right frame has a frame body and a lens, the frame body forming a flange thereon. The hard enveloping frames abut against sides of the flanges, and form positioning portions on inward sides thereof, and connecting devices on outward sides thereof. The soft pads are respectively assembled on the flanges of the left frame and the right frame, and are unitarily connected by a connecting portion. Each soft pad includes a touch portion for touching a user's face, and an assembling portion for covering the flange. The head strap is assembled on the connecting devices of the hard enveloping frames. The swimming goggles are worn comfortably and suit for users with different face profiles. At the same time, the swimming goggles prevent from leakage.

### BACKGROUND OF THE INVENTION

1. Field of the Invention

The present invention relates to swimming goggles, and particularly to swimming goggles which are worn comfortably and which are not subject to a user's pulling force, and therefore prevent from water leakage.

2. Related Art

In general, swimming goggles are divided into two types: the first is separate type, and the second is integral type. The separate type of swimming goggles have a separate connecting member to connect a left frame and a right frame; while the integral type of swimming goggles have a left frame and a right frame integrally formed with a connecting part. The instant invention is involved of the separate type of swimming goggles. The connecting member of the separate type is ordinarily made of hard material, and therefore, is not flexible when being worn. Accordingly, such swimming goggles cannot suit for users with different nose profiles, and are even rather uncomfortable for the users with Roman nose. Due to lack of flexibility, the left frame and the right frame can not fit to each other closely, taking risk of leakage.

### SUMMARY OF THE INVENTION

Accordingly, an object of the present invention is to provide swimming goggles which are worn comfortably and prevent from leakage effectively. The swimming goggles have a pad for touching a user's face and a connecting member for connecting a left frame and a right frame. The pad, the left frame and the right frame are all made of soft material, which touch a user's face comfortably, and fit for users with different nose profiles.

The swimming goggles comprise a left frame, a right frame, hard enveloping frames, soft pads and a head strap. Each of the left frame and the right frame has a frame body and a lens, the frame body forming a flange thereon. The hard enveloping frames abut against sides of the flanges, and form positioning portions on inward sides thereof, and connecting devices on outward sides thereof. The soft pads are respectively assembled on the flanges of the left frame and the right frame, and are unitarily connected by a connecting portion. Each soft pad includes a touch portion for touching a user's face, and an assembling portion for covering the flange. The head strap is assembled on the connecting devices of the hard enveloping frames. In assembly, the flanges of the left frame and the right frame are assembled on the assembling portions of the soft pads. The hard enveloping frames are respectively assembled on the left frame and the right frame. The both ends of the connecting portion are respectively assembled on the positioning portions of the hard enveloping frames.

The swimming goggles further comprise strap adjusting devices. Each strap adjusting device includes an operating plate and a cover. The operating plate has a branch and a stop arm. The branch is pivoted by leverage on second axis holes of the bases. The cover is assembled on the base and defines an opening corresponding to the operating plate. The stop arm is engaged with or disengaged from the head strap by operating the operating plate.

### BRIEF DESCRIPTION OF THE DRAWINGS

FIG. 1 is an exploded view of swimming goggles of the present invention.

FIG. 2 is an assembled view of the swimming goggles of FIG. 1.

FIG. 3 is a front view of the swimming goggles of FIG. 2.

FIG. 4 is a cross-sectional view taken along the line 4-4 in FIG. 3.

FIG. 5 is a cross-sectional view taken along the line 5-5 in FIG. 3.

FIGS. 6A, 6B, 7A-7C and 8A-8E illustrate the swimming goggles being assembled step by step.

FIG. 9 is a partially enlarged view of the swimming goggles, especially showing a head strap thereof being adjusted.

### DESCRIPTION OF THE PREFERRED EMBODIMENTS

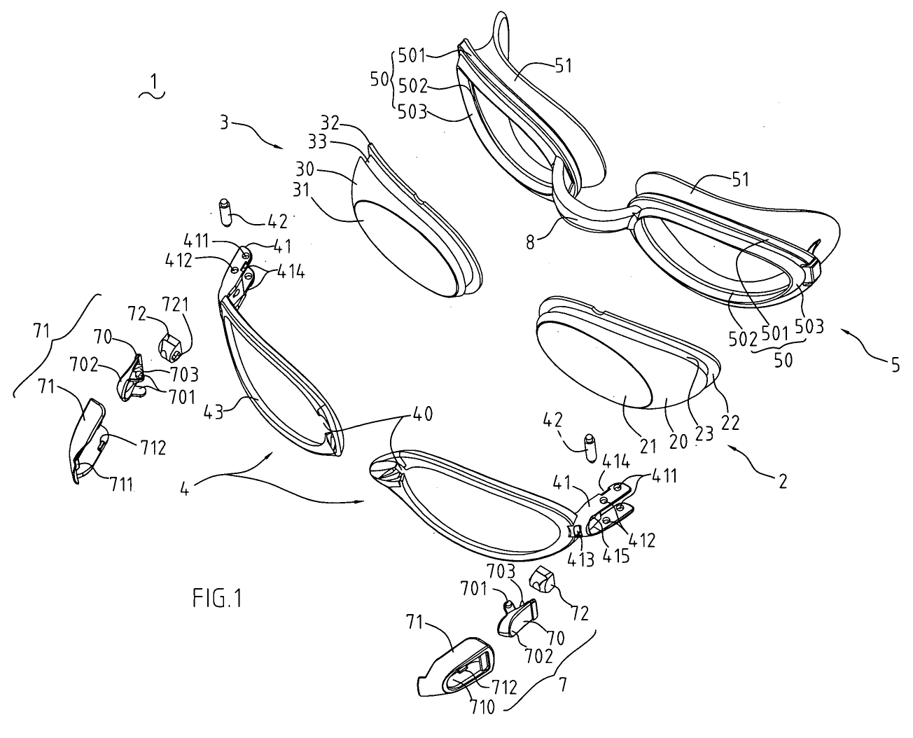

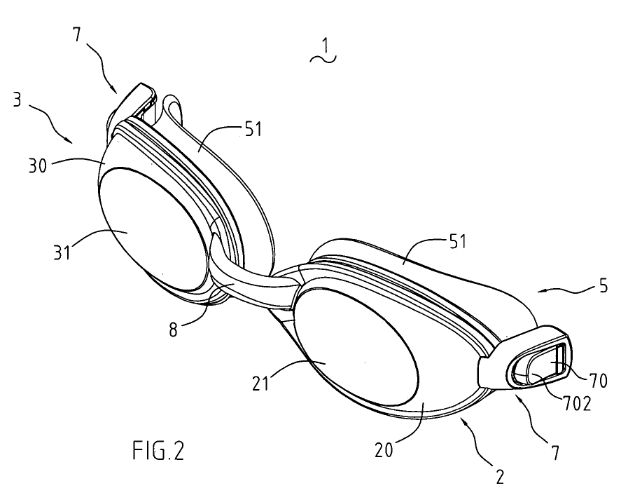

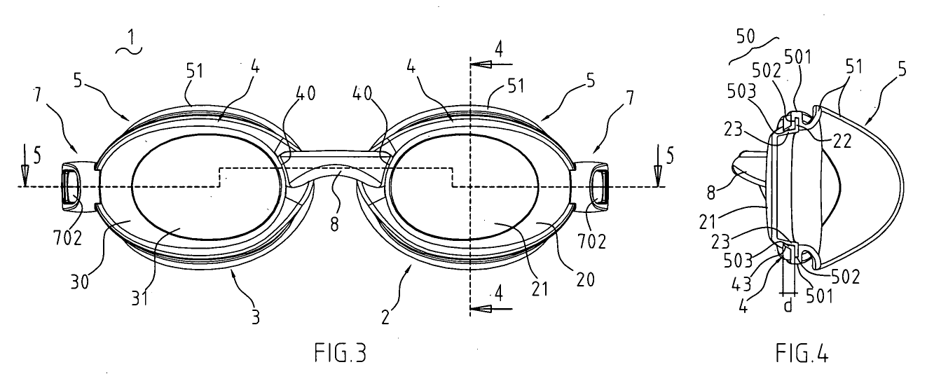

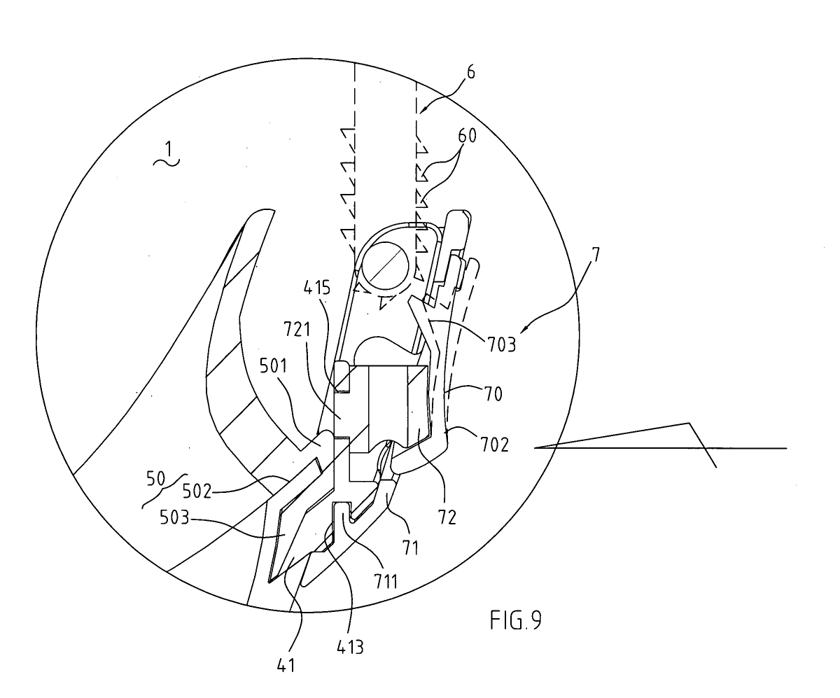

With reference to FIGS. 1 through 3, swimming goggles 1 in accordance with the present invention comprise a left frame 2, a right frame 3, hard enveloping frames 4, soft pads 5, a head strap 6 (see FIG. 9) and strap adjusting devices 7. The left frame 2 and the right frame 3 are of the same structure, and are both made of PP (Polypropylene). The left frame 2 (the right frame 3) comprises a frame body 20 (30) and an elliptic lens 21 (31) which are integrally formed with hard material. A flange 22 (32) is unitarily extended from an edge of the frame body 20 (30). An annular embedding groove 23 (33) is defined along the frame body 20 (30) and in the vicinity of the flange 22 (32).

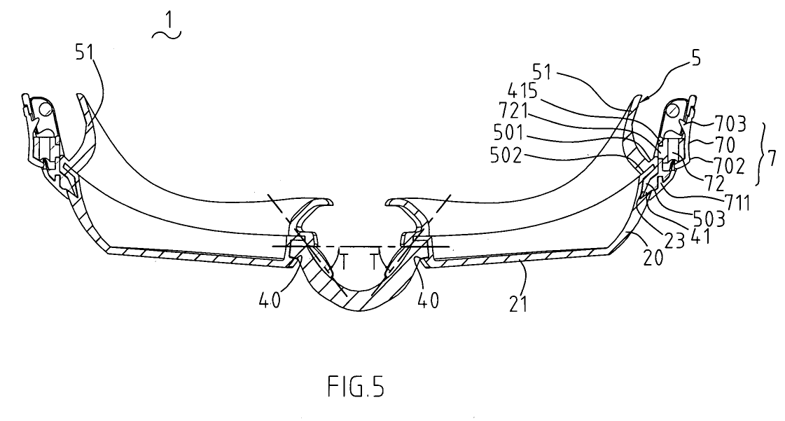

The hard enveloping frames 4 are assembled on and abut against sides of the flanges 22, 32, and form positioning portions on inward sides thereof. According to an embodiment of the present invention, the positioning portions include opening 40 in inward sides of the hard enveloping frames 4. The parts around the opening 40 substantially extend upward and slantwise to form angles T (referring to FIG. 5). The hard enveloping frames 4 form connecting devices on outward sides thereof. Each connecting device comprises an integral base 41, and a positioning post 42 extending into first axis holes 411 of the base 41 for supporting the head strap 6. The hard enveloping frames 4 have sloped inward sides 43, as shown in FIG. 4.

The strap adjusting devices 7 are mounted on the bases 41. Each strap adjusting device 7 comprises an operating plate 70, a cover 71 and a resilient portion 72. The operating plate 70 forms a branch 701 pivoted by leverage on second axis holes 412 of the bases 41. The operating plate 70 has a manual end 702 slightly beyond the cover 71 when assembled for manual operation. The operating plate 70 forms a stop arm 703 for abutting against a stop groove 60 (referring to FIG. 9) of the head strap 6. The cover 71 is assembled on the base 41, and defines an opening 710 corresponding to the operating plate 70 for receiving the operating plate 70. Recesses 413, 414 are respectively defined in a front and an inward side of the base 41, and projections 711, 712 are respectively formed on a front and an inward side of the cover 71 for cooperating with the recesses 413, 414, thereby assembling the cover 71 and the base 41 together. The resilient portion 72 forms a neck portion 721 on a bottom thereof. The base 41 defines an embedding hole 415 corresponding to the manual end 702 of operating plate 70 for assembling the neck portion 721, thereby fixing the resilient portion 72. When assembled, the resilient portion 72 extends below the manual end 702 of the operating plate 70 for providing return force.

The soft pads 5 are made of Thermal Plastic Rubber (TPR), and are respectively assembled on the flanges 22, 32 of the left frame 2 and the right frame 3. The soft pads 5 are unitarily connected by a connecting portion 8. Each soft pad 5 comprises an assembling portion 50 for covering the flange 22, 32, and a touch portion 51 for touching a user's face. Referring to FIG. 4, the assembling portion 50 includes an annular body 501 and an abutting surface 503. A passageway 502 is defined in an inward side of the annular body 501 for receiving the flange 22(32). The abutting surface 503 extends unitarily from a side of the annular body 501 and is inclined relative to the inward side 43 of the hard enveloping frame 4 for abutting firmly. The touch portion 51 extends unitarily from the annular body 501 and toward a side of the abutting surface 503 for touching a user's face comfortably.

Further referring to FIG. 4, it is noted that, the embedding groove 23 has a width d slightly less than the side width of the hard enveloping frame 4 and the annular body 501 such that in assembly, the hard enveloping frame 4 tightly presses against the abutting surface 503 and a side of the annular body 501 upon the embedding groove 23. Referring to FIG. 5, the angles T of the hard enveloping frames 4 appropriately restrain flexibility of the connecting portion 8, avoiding excessive deformation when being worn, thereby fitting for a user's nose bridge.

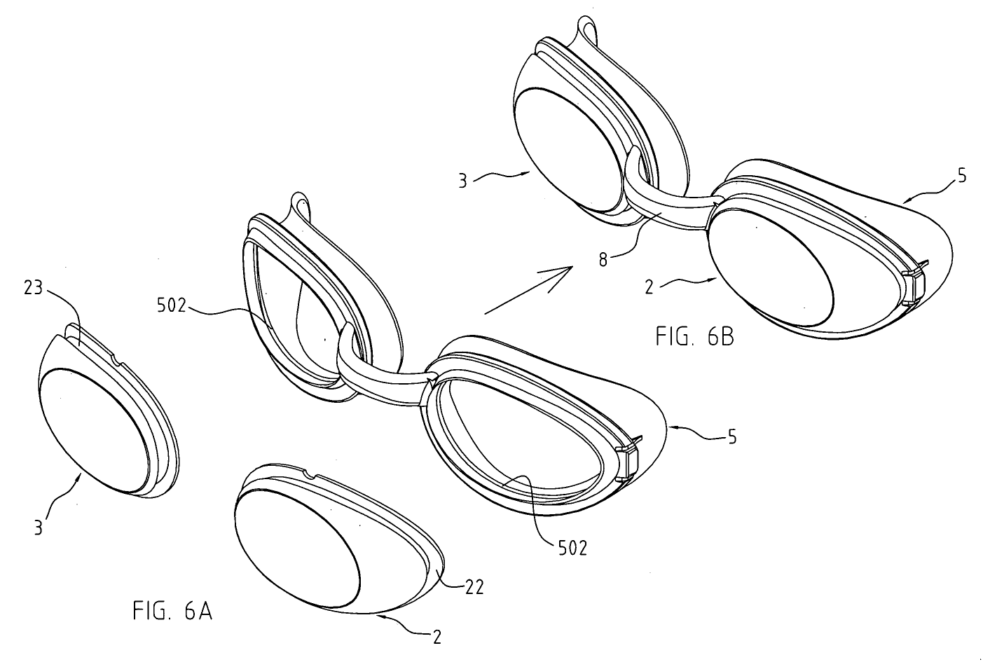

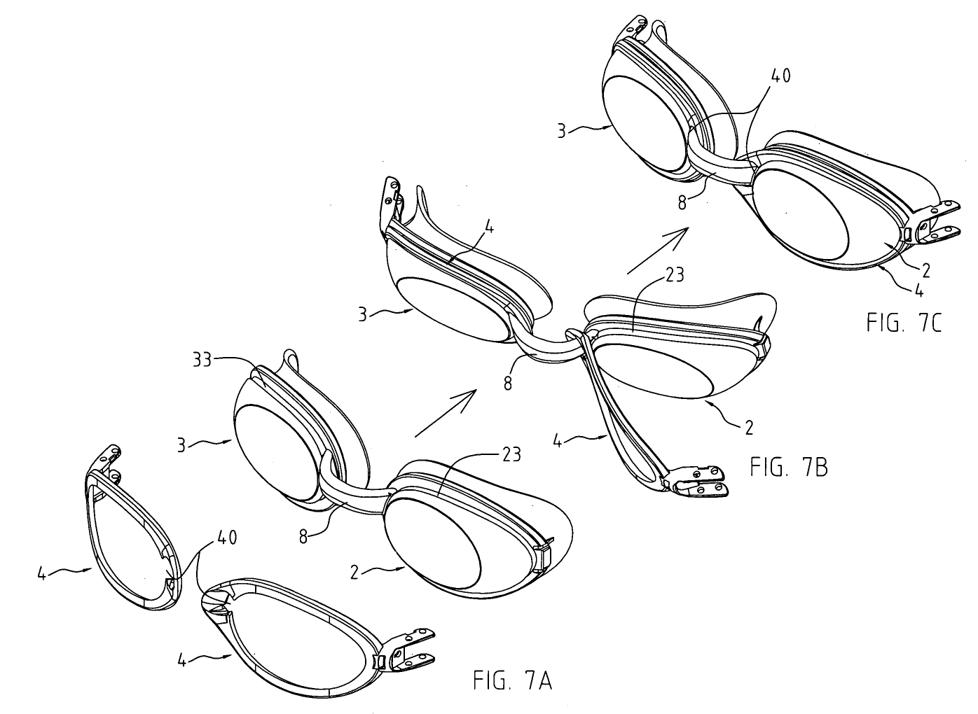

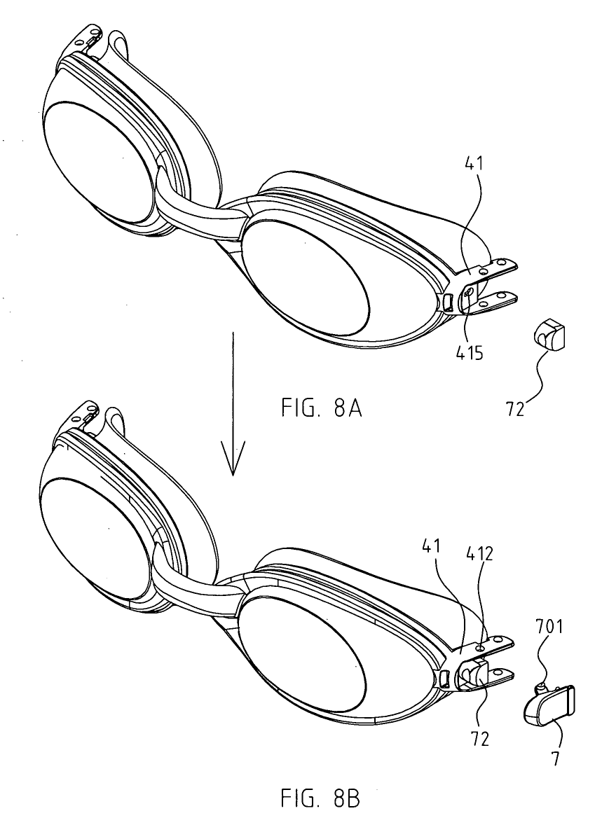

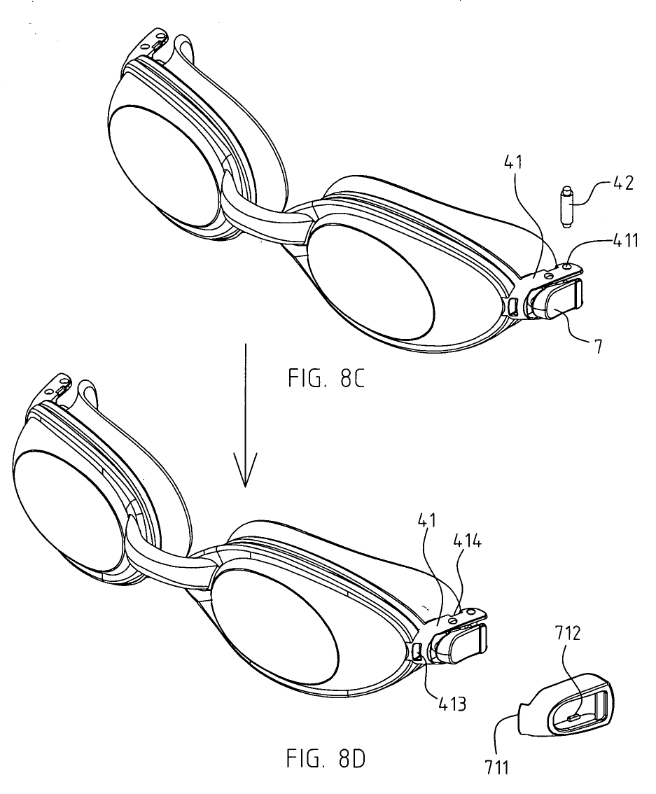

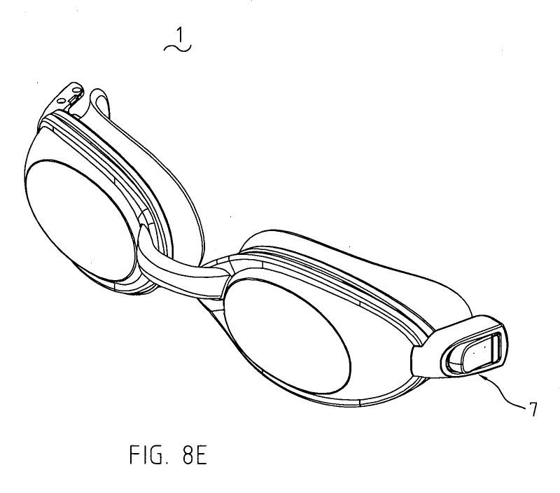

In assembly, as shown in FIGS. 6-8, the flanges 22, 32 of the left frame 2 and the right frame 3 are received in the passageways 502 of the soft pads 5 (see FIGS. 6A and 6B). The hard enveloping frames 4 are respectively assembled in the embedding grooves 23, 33 of the left frame 2 and the right frame 3. Both ends of the connecting portion 8 are respectively assembled in the opening 40 of the hard enveloping frames 4 (see FIGS. 7A, 7B and 7C). The resilient portions 72 are mounted on the embedding holes 415 of the bases 41 (see FIG. 8A). The branches 701 of the operating plates 7 are pivoted onto the second axis holes 412 of the bases 41 (see FIG. 8B). The positioning posts 42 are assembled on the first axis holes 411 of the bases 41 (see FIG. 8C). The projections 711, 712 of the covers 72 are assembled on the recesses 413, 414 of the bases 41 (see FIG. 8D). Thus, the swimming goggles 1 of the present invention are assembled reliably (see FIG. 8E).

Further referring to FIG. 9, when the swimming goggles 1 of the present invention are worn, the head strap 6 can be adjusted by manipulating the manual ends 702 of the operating plates 7 (as shown by the arrow in FIG. 9). When the stop arms 703 disengage from the stop grooves 60 of the head strap 6, the head strap 6 is adjustable in length. Meanwhile the resilient portions 72 are pressed and reserve energy. Once released, the operating plates 7 return back by the reserved energy of the resilient portions 72.

It is understood that the invention may be embodied in other forms without departing from the spirit thereof. Thus, the present examples and embodiments are to be considered in all respects as illustrative and not restrictive, and the invention is not to be limited to the details given herein.

What is claimed is:

1. Swimming goggles comprising:
a left frame and a right frame, each of the left frame and the right frame having a frame body and a lens, the frame body forming a flange thereon;
hard enveloping frames abutting against sides of the flanges, and forming positioning portions on inward sides thereof, and connecting devices on outward sides thereof;
soft pads being respectively assembled on the flanges of the left frame and the right frame, and be unitarily connected by a connecting portion, each soft pad including a touch portion for touching a user's face, and an assembling portion for covering the flange; and
a head strap being assembled on the connecting devices of the hard enveloping frames.

2. The swimming goggles as claimed in claim 1, wherein the frame body and the lens of each of the left frame and the right frame are integrally formed with hard material.

3. The swimming goggles as claimed in claim 2, wherein the positioning portions include opening in inward sides of the hard enveloping frames.

4. The swimming goggles as claimed in claim 3, wherein parts around the opening substantially extend upward to form angles.

5. The swimming goggles as claimed in claim 4, wherein the assembling portion of each soft pad includes an annular body and an abutting surface extending unitarily from a side of the annular body, a passageway being defined in an inward side of the annular body.

6. The swimming goggles as claimed in claim 5, wherein the touch portion of each soft pad extends unitarily from the annular body and toward a side of the abutting surface.

7. The swimming goggles as claimed in claim 6, wherein the hard enveloping frames have sloped inward sides, and wherein the abutting surfaces of the soft pads are inclined relative to the inward sides of the hard enveloping frames for abutting firmly.

8. The swimming goggles as claimed in claim 6, wherein an annular embedding groove is defined along the frame body and in the vicinity of the flange, the embedding groove having a width slightly less than side width of the hard enveloping frame and the annular body such that in assembly, the hard enveloping frame tightly presses against the abutting surface and a side of the annular body upon the embedding groove.

9. The swimming goggles as claimed in claim 1, wherein each of connecting devices comprises a base defining first axis holes and second axis holes, a positioning post extending into the first axis holes of the base.

10. The swimming goggles as claimed in claim 9, further comprising strap adjusting devices, each strap adjusting device including an operating plate and a cover, the operating plate having a branch and a stop arm, the branch being pivoted by leverage on second axis holes of the base, the cover being assembled on the base and defining an opening corresponding to the operating plate, the stop arm being engaged with or disengaged from the head strap by operating the operating plate.

11. The swimming goggles as claimed in claim 10, wherein the head strap defines a plurality of stop grooves therein.

12. The swimming goggles as claimed in claim 10, further comprising resilient portions below a manual end of the operating plate for providing return force.

13. The swimming goggles as claimed in claim 12, wherein the resilient portion forms a neck portion on a bottom thereof, and the base defines an embedding hole corresponding to the manual end of operating plate for assembling with the neck portion thereby fixing the resilient portion.

14. The swimming goggles as claimed in claim 10, wherein recesses are respectively defined in a front and an inward side of the base, and projections are respectively formed on a front and an inward side of the cover for cooperating with the recesses, thereby assembling the cover and the base together.
